(19) United States
(12) Patent Application Publication
(10) Pub. No.: US 2007/0260208 A1
(43) Pub. Date: Nov. 8, 2007

(54) Urine collection bag supporting device

(76) Inventor: Dean May, Muncy, PA (US)

Correspondence Address: JAMES RAY& ASSOCIATES, 2640 Pitcairn Road, Monroeville, PA, 15146, US

(21) Appl. No.: 11/796,348

(22) Filed: Apr. 27, 2007

Related U.S. Application Data
(60) Provisional application No. 60/797,193, filed on May 3, 2006.

(51) Int. Cl. A61F 5/44

(52) U.S. Cl. 604/345; 604/353; 604/544

(57) ABSTRACT

A support bag for use with a urine collection bag includes a bag body sized to receive the urine collection bag and a plurality of attachment devices, any one of which could be used to attach the bag body to the patient's clothing. The bag body has windows through which a urine drain line and straps from the urine collection bag will extend. The attachment device includes a hook, a strap, and a length of cord.

The applicant claims priority from his provisional application filed on May 3, 2006 and assigned Ser. No. 60/797,193. The present invention relates to a retaining bag for retaining a urine collection bag worn under the clothing of a patient.

### BACKGROUND OF THE INVENTION

Urine collection bags are used by mobile incontinent patients. Male patients use a urine collection bag of the type having an inlet port at the upper end thereof, an outlet port at the lower end thereof, and a plurality of straps that are suitable for retaining the bag to a leg of the patient. Such urine collection bags are made of plastic that is uncomfortable against the skin. For a patient who is mobile and using his legs, the straps provided with such urine collection bags tend to slide downwardly along the leg as the patient moves causing distress to the wearer.

Efforts have been made to provide a support apparatus for assisting in maintaining a urine collection bag to the body of a wearer, but such prior efforts have not been totally successful. Many of the existing bags require the attachment of an upper end of the bag to a belt that is strapped around the wearer's waist. Where a patient has body contours, or other conditions, that render it uncomfortable or impossible to wear a belt, support device that require the use of a belt are inadequate. Patients who rely on suspenders to retain their pants are typical incapable of employing a belt-type retention device. Accordingly, there is a need for a device for holding a urine collection bag for a patient who cannot rely on a belt to support a urine collection bag.

### SUMMARY OF THE INVENTION

The present invention is embodied in a support bag for retaining the urine collection bag worn by a patient, the urine collection bag being of the type having a urinary catheter inlet at the top, a drainage outlet at the bottom and a plurality of straps extending from the sides for retaining the collection bag to the leg of a patient. The invention is particularly useful for a patient who cannot, or prefers not to wear a belt around his or her waist to suspend pants and the like.

The support bag has a bag body having side walls defining a sleeve, a bottom, and an open upper end, and is sized to receive a commonly used urine collection bag. The bag body has a window in the bottom thereof through which the drainage outlet of the urine collection bag can extend such that the urine collection bag can be drained while being retained within the support bag. The support bag further has a plurality of apertures, or windows, in the side wall each of which is positioned to receive one of the straps of the urine collection bag. The support bag further has an attachment device at the upper end of the bag body for attaching the support bag to the waistband of a pair of trousers or to any other piece of clothing worn by the patient.

When in use, the urine collection bag is inserted into the bag body with the drainage outlet extending through the window in the bottom thereof. The straps of the urine collection bag are extended through the windows in the side walls and are fastened around a leg of the patient. Thereafter, the attachment device at the upper end of the bag is attached to the waistband of the patient's pants to thereby support the weight of the urine in the urine collection bag.

The bag body is made of a material that will not be irritating to the skin of the wearer. Preferably, the bag body is made of canvas, or other durable, flexible material. Also, the bag body is preferably provided with a plurality of alternative attachment devices, any one of which is attachable to the bag body and is suitable for retaining the weight of the urine collection bag and the urine therein. Any one of the attachment devices can be connected or disconnected to a connector at the upper end of the bag body. The attachment devices include an elongate strap that can be wrapped around a belt, should a belt be available, a hook that can be hooked over a waistband, and a cord, the free end of which can be looped around a button, a suspender holder, a belt loop, or the like. The hook for hooking over the waistband of a wearer is preferably made of a plastic or metal and has flexibility and a memory for springing back to its original configuration. The clip is configured to compress tightly around the fabric that makes up the waistband of a pair of pants.

### BRIEF DESCRIPTION OF THE DRAWINGS

A better understanding of the device will be had after a reading of the following detailed description taken in conjunction with the drawings wherein:

FIG. 1 is a front elevational view of a support bag in accordance with the invention;

FIG. 2 is a second front elevational view of the support bag shown in FIG. 1 with a urine collection bag therein;

FIG. 3 is a cross-sectional view of the support bag and urine collection bag taken through line 3-3 of FIG. 2 showing the configuration of the parts while the urine collection bag is at least partially full of urine;

FIG. 4 is a front elevational view showing a plurality of attachment devices, any one of which may be used with the support bag shown in FIG. 1; and

FIG. 5 is a side view of the clip shown in FIG. 4.

### DETAILED DESCRIPTION OF PREFERRED EMBODIMENT

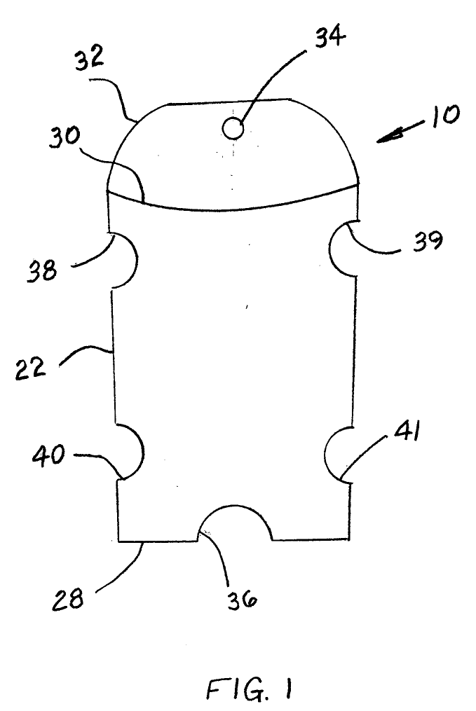

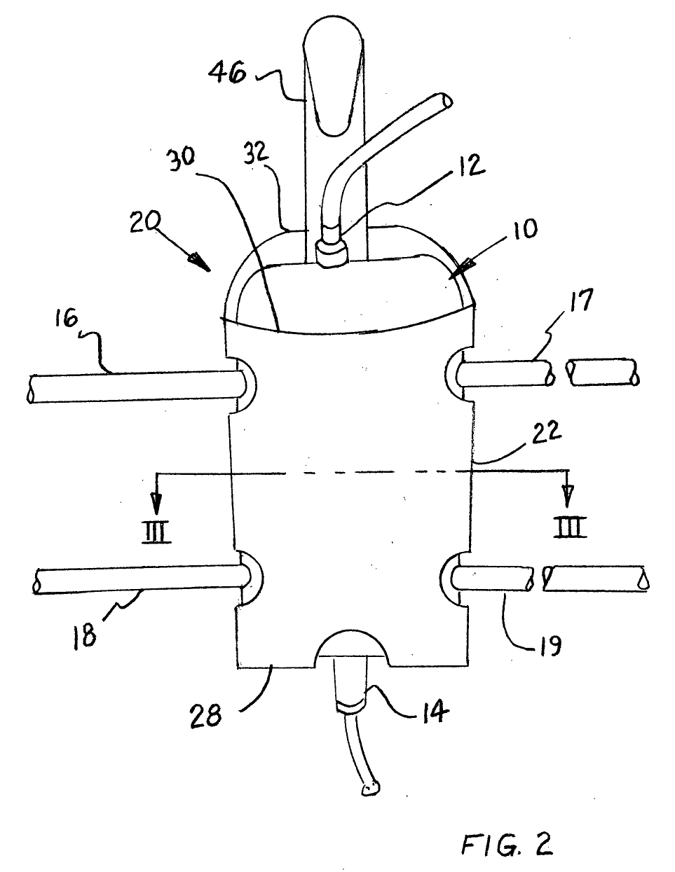

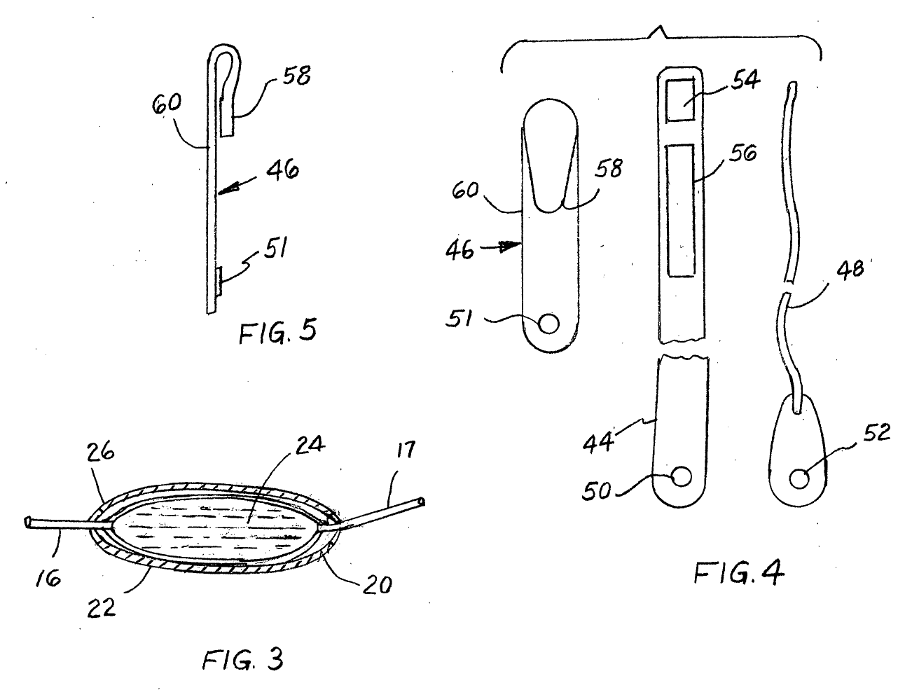

Referring to FIGS. 1, 2, and 3, a urine collection bag 10 of the type provided for incontinent patients is made of a flexible plastic material with the uninflated bag being generally rectangular in shape. The collection bag 10 has a urinary catheter inlet 12 at the upper end thereof, a drainage outlet 14 at the bottom end thereof, and a plurality of straps 16, 17, 18, 19 extending from the sides thereof with straps 16 and 17 extending from opposite sides of the upper end of the bag 10 and straps 18 and 19 extending from opposite sides of the lower end of the bag 10. The straps 16-19 are adapted to fit around the leg of a user, not shown, for attaching the collection bag 10 to the user's leg.

I have found that the straps 16-19 are inadequate to retain the collection bag 10 to a user's leg, especially when the bag 10 is at least partially filled with urine. Gradually, the straps 16-19 will slide downwardly along the user's leg causing concern to the user and requiring the user to be constantly aware of the condition of the straps 16-19. Furthermore, the plastic of which the collection bag 10 is made is uncomfortable when applied to the skin of a user. To improve the comfort of one using a collection bag 10, a support bag 20 in accordance with the invention is provided. The support bag 20 has a bag body 22 made of canvas or some other flexible material that would be comfortable when used against the wearer's skin and suitable for retaining the weight of a urine collection bag 10 when it has a quantity of urine 24 therein. The central portion of the bag body 22 has flexible side walls 26 forming a sleeve, the inner opening of which is sufficiently large to receive the side walls of the urine collection bag 10, and a bottom 28 extending across the lower end of the side walls 26 to thereby retain the weight of urine in the urine collection bag 10. The bag body 22 has an open upper end 30 with an upwardly directed extension 32 to a portion of the side walls 26. The extension 32 has a connector 34 thereon for removably connecting to a complementary connector or an attachment device for attachment to an item of clothing worn by the patient. The connector 34 may be one part of a two part snap connector of the type well known in the art, or may be in the form of a button or may be one portion of a loop and hook connector of the type sold under the trademark Velcro.

The bottom 28 of the bag body 22 has an aperture, or window 36 therein sized and positioned to receive the catheter outlet 14 is the urine collection bag 10. The side walls 26 of the bag body 22 also have a plurality of windows 38, 39, 40, 41 therein, with each of the windows 38-41 positioned for receiving one of the straps 16-19 of the collection bag 10. Accordingly, the urine collection bag 10 can be inserted into the pocket of the bag body 22 with the drainage outlet 14 extending through the bottom window 36 and each of the straps 16-19 extending through one of the aligned windows 38-41 of the bag body 22 thereby allowing the straps 16-19 to be positioned around the leg of the patient. It should be appreciated that the windows 38-41 are not mere slits in the bag body 22, but cut out portions, thereby allowing the support bag 20 to move with respect to the urine collection bag 10 as the collection bag 10 fills with urine 24. By providing cut out windows 38-41, the fabric of the bag body 22 can remain against the skin of the wearer without causing uncomfortable bunching or pinching as the bag inflates with urine or is subsequently deflated using the outlet 14.

Referring to FIGS. 1, 4, and 5, the support bag 20 is retained to the waistband, not shown, of a patient wearing a pair of pants by an attachment device such as a flexible strap 44, a plastic or metal clip 46, or a cord 48. Each of the attachment devices 44, 46, 48 has a connector 50, 51, 52 at the lower end thereof that is complementary to the connector 34 on the bag body 22. Accordingly, if connector 34 is a snap type connector, then connectors 50-52 are complementary snap connectors and, if connector 34 is a loop and hook type connector, then connectors 50-52 are the complementary member of the loop and hook connector, and if connector 34 is a button, then connectors 50-52 are loops that will fit over the button.

For strap 44, the end thereof opposite the connector 50 has a small strip of one member 54 of a loop and hook type connector at the distal end thereof. Adjacent the member 54 on strap 44, and toward the connector 50 is a somewhat longer strip of the second member 56 of the loop and hook type connector. Accordingly, the free end of the strap 44 can be wrapped around a belt or other portion of clothing and the members 54, 56 assembled to each other to retain the support bag 20 and the urine collection bag 10. The upper end of the clip 46 includes an inverted U-shaped hook having a hook end 58 that presses against a hook base 60 thereof for compressing around the fabric of a waistband of a pair of pants or the like. The hook end 58 and base 60 are made of a generally rigid material with spring qualities and a memory for returning to a predetermined shape, as for example, a spring steel. The cord 48 has sufficient strength to bear the weight of the urine collection bag 10 and its contents and has sufficient length to allow the distal end 62 thereof to be wrapped around a button or belt loop to thereby retain the weight of the collection bag 10.

While the present invention has been described with respect to a single embodiment, it will be appreciated that many modifications and variations may be made without departing from the spirit and scope of the invention. It is therefore the intent of the appended claims to cover all such modifications and variations that fit within the spirit and scope of the invention.

What is claimed:

1. A support bag for retaining a urine collection bag worn by a patient wherein said urine collection bag has a urinary catheter inlet at the top thereof, a drainage outlet at the bottom thereto, and a plurality of straps extending from sides thereof, said straps for retaining said collection bag to a leg of said patient, and wherein said patient is wearing clothing having a waistband, said support bag comprising
a flexible bag body having side walls, a bottom, and an open upper end,
said bag body sized to receive said collection bag within said side walls while said collection bag is full of urine,
a window in said bottom of said bag body for receiving said drainage outlet,
a plurality of windows in said side walls of said bag body wherein each of said windows will receive one of said plurality of straps of said urine collection bag and wherein said each of said plurality of straps will extend through one of said windows for retaining said support bag and said collection bag to said patient's leg, and
an attachment device for removably attaching said upper end to said waistband.

2. The support bag of claim 1 wherein said plurality of windows in said side walls are cut out portions of said side walls.

3. The support bag of claim 1 wherein said attachment device is a hook for hooking over said waistband.

4. The support bag of claim 3 wherein said hook is made of a generally rigid material with spring qualities and a memory for returning to a predetermined shape and includes a clip end that compresses towards a clip base.

5. The support bag of claim 1 wherein said attachment device is a cord for tying to a portion of said clothing.

6. The support bag of claim 1 and further comprising
a connector at said open upper end,
and wherein said attachment device is one of a plurality of attachment devices having a connector end for engaging said connector and an attachment end for attaching to a portion of said clothing.

7. The support bag of claim 6 wherein one of said attachment devices is a hook.

8. The support bag of claim 6 wherein one of said attachment devices is a cord.

9. The support device of claim 6 wherein one of said attachment devices includes a hook and loop type connector on an end of a strap for connecting around a portion of said clothing.
